(19) United States
(12) Patent Application Publication
(10) Pub. No.: US 2020/0375721 A1
(43) Pub. Date: Dec. 3, 2020

(54) IMPLANTABLE DAMPING DEVICE FOR MODIFYING BLOOD FLOW CHARACTERISTICS

(71) Applicant: The Brain Protection Company PTY LTD, Paddington (AU)

(72) Inventors: David Stephen Celermajer, Vaucluse (AU); Anthony John Ujhazy, East Lindfield (AU); Zoran Milijasevic, Bayview (AU)

(21) Appl. No.: 16/760,985

(22) PCT Filed: Nov. 2, 2018
(86) PCT No.: PCT/AU2018/051191

§ 371 Date: May 1, 2020

(30) Foreign Application Priority Data
Nov. 3, 2017 (AU) 2017904479

(51) Int. Cl. A61F 2/06; A61F 2/88; A61F 2/958

(52) U.S. Cl. CPC A61F 2/06; A61F 2/88; A61F 2002/825; A61F 2002/068; A61F 2/958

(57) ABSTRACT

An implantable damping device (100) for modifying blood flow characteristics in a vessel (50), the device (100) including: a first expansion chamber (110) having a first chamber proximal end (130), a first chamber distal end (170) and a first chamber intermediate portion (140) having a larger cross-sectional area than the first chamber proximal and distal ends (130), wherein the first expansion chamber (110) generates a pressure drop in blood flow downstream of the device (100) relative to blood flow upstream of the device (100).

### TECHNICAL FIELD

The present disclosure relates to an implantable damping device for modifying blood flow characteristics. In particular, the present invention relates to an implantable damping device for treating an artery.

### BACKGROUND OF THE INVENTION

The heart supplies oxygenated blood to the body through a network of interconnected, branching arteries starting with the largest artery in the body, the aorta. As shown in the schematic view of the heart and selected arteries in FIG. 1A, the portion of the aorta closest to the heart is divided into three regions: the ascending aorta (where the aorta initially leaves the heart and extends in a superior direction), the aortic arch, and the descending aorta (where the aorta extends in an inferior direction). Three major arteries branch from the aorta along the aortic arch: the brachiocephalic artery, the left common carotid artery, and the left subclavian artery. The brachiocephalic artery extends away from the aortic arch and subsequently divides into the right common carotid artery, which supplies oxygenated blood to the head and neck, and the right subclavian artery, which predominantly supplies blood to the right arm. The left common carotid artery extends away from the aortic arch and supplies the head and neck. The left subclavian artery extends away from the aortic arch and predominantly supplies blood to the left arm. Each of the right common carotid artery and the left common carotid artery subsequently branches into separate internal and external carotid arteries.

The descending aorta extends downwardly, and defines the descending thoracic aorta and subsequently the abdominal aorta before branching into the left and right iliac arteries. Various organs of the body are supplied by arteries which junction with and are supplied by the descending aorta.

In many people, at junctions where the abdominal aorta connects with other arteries, such as the renal arteries, hepatic artery and splenic artery, there may be a significant step-down in diameter of the abdominal aorta. This rapid reduction in diameter is thought to be responsible for undesirable reflected pressure waves which may return upstream toward the heart.

During the systole stage of a heartbeat, contraction of the left ventricle forces blood into the ascending aorta that increases the pressure within the arteries (known as systolic blood pressure). The volume of blood ejected from the left ventricle creates a pressure wave, known as a pulse wave, which propagates through the arteries propelling the blood. The pulse wave causes the arteries to dilate. When the left ventricle relaxes (the diastole stage of a heartbeat), the pressure within the arterial system decreases (known as diastolic blood pressure), which allows the arteries to contract.

The difference between the systolic blood pressure and the diastolic blood pressure is the “pulse pressure,” which generally is determined by the magnitude of the contraction force generated by the heart, the heart rate, the peripheral vascular resistance, and diastolic “run-off” (e.g., the blood flowing down the pressure gradient from the arteries to the veins), amongst other factors. High flow organs, such as the brain, are particularly sensitive to excessive pressure and flow pulsatility. Other organs such as the kidneys, liver and spleen may also be damaged over time by excessive pressure and flow pulsatility.

To ensure a relatively consistent flow rate to such sensitive organs, the walls of the arterial vessels expand and contract in response to the pressure wave to absorb some of the pulse wave energy. As the vasculature ages, however, the arterial walls lose elasticity, which causes an increase in pulse wave speed and wave reflection through the arterial vasculature. Arterial stiffening impairs the ability of the carotid arteries and other large arteries to expand and dampen flow pulsatility, which results in an increase in systolic pressure and pulse pressure. Accordingly, as the arterial walls stiffen over time, the arteries transmit excessive force into the distal branches of the arterial vasculature.

Research suggests that consistently high systolic pressure, pulse pressure, and/or change in pressure over time (dP/dt) increases the risk of dementia, such as vascular dementia (e.g., an impaired supply of blood to the brain or bleeding within the brain). Without being bound by theory, it is believed that high pulse pressure can be the root cause or an exacerbating factor of vascular dementia and age-related dementia (e.g., Alzheimer's disease). As such, the progression of vascular dementia and age-related dementia (e.g., Alzheimer's disease) may also be affected by the loss of elasticity in the arterial walls and the resulting stress on the cerebral vessels. Alzheimer's disease, for example, is generally associated with the presence of neuritic plaques and tangles in the brain. Recent studies suggest that increased pulse pressure, increased systolic pressure, and/or an increase in the rate of change of pressure (dP/dt) may, over time, cause microbleeds within the brain that may contribute to the neuritic plaques and tangles.

### SUMMARY OF THE INVENTION

Several aspects of the present technology provide an implantable damping device for modifying blood flow characteristics in a vessel, the device including:
at least a first expansion chamber having a first chamber proximal end, a first chamber distal end and a first chamber intermediate portion having a larger cross-sectional area than the first chamber proximal and distal ends,
wherein the first expansion chamber generates a pressure drop in blood flow downstream of the device relative to blood flow upstream of the device.

The implantable device can further comprise a second expansion chamber having a second chamber proximal end, a second chamber distal end and a second chamber intermediate portion having a larger cross-sectional area than the second chamber proximal and distal ends.

The distal end of the first expansion chamber and the proximal end of the second expansion chamber can be longitudinally connected and in fluid communication.

Several embodiments of the first and second chamber intermediate portions each have a larger diameter than a native diameter of the vessel.

The first and second chamber intermediate portions can be cylindrical and of equal diameter.

The first and second expansion chambers can each have a cross-sectional profile in the form of a truncated ellipse when viewed through a plane which is parallel to a longitudinal axis.

A wrap can be wrapped externally around a portion of the vessel at a location which is radially external to a portion of the device, and the wrap can be configured to radially compress the vessel, such that the vessel locally abuts against the damping device.

The wrap generally extends longitudinally between the first chamber intermediate portion and the second chamber intermediate portion.

The wrap can be helical.

The implantable device can further comprise a plurality of longitudinally extending spines extending between the first chamber proximal end and the second chamber distal end.

At or near the first chamber intermediate portion and/or the second chamber intermediate portion, the spines can be secured to one or more radially expansible/collapsible band.

The band can be defined by a plurality of struts, and the struts can have a zig zag profile when viewed in a plane extending parallel to a longitudinal axis.

At the proximal end of the first expansion chamber, each spine can be connected to an adjacent spine, and at the distal end of the second expansion chamber the spine is connected to a different adjacent spine.

At the proximal end of the first expansion chamber and the distal end of the second expansion chamber, each spine can be connected to a ring.

A tubular region of constant diameter can be located at the proximal end of the first expansion chamber and the distal end of the second expansion chamber.

The spines can be defined by a continuous wire.

Several aspects of the present technology are directed to implantable damping devices for modifying blood flow characteristics in a vessel, including:

an expansion chamber having a proximal end and a distal end, wherein an internal cross-section of a blood flow passage within the implantable device gradually decreases between the proximal end and the distal end.

The implantable damping device can further comprise a plurality of longitudinally extending spines extending between the proximal and distal ends, wherein at the proximal end, the spines are secured to a radially expansible/collapsible band.

The band can be defined by a plurality of struts, and the struts can have a zig zag profile when viewed in a plane extending parallel to a longitudinal axis.

At the distal end, each spine can be connected to one adjacent spine.

Several aspects of the present technology are directed to methods of adjusting a cross sectional area of the implantable damping device as described above by:
inserting a balloon into the implantable damping device; and
expanding the balloon to expand the implantable damping device between a first cross-sectional size and a larger second cross-sectional size.

### BRIEF DESCRIPTION OF THE DRAWINGS

Several specific embodiments of the present technology are described below by way of specific examples with reference to the accompanying drawings, in which:

FIG. 1A is a schematic illustration of a human heart and a portion of the arterial system near the heart;

FIG. 1B is a perspective view of a blood flow modifying device according to the present technology;

FIG. 2 is a schematic view of the blood flow modifying device of FIG. 1B depicted within a vessel;

FIG. 3 is a perspective view of a blood flow modifying device according to the present technology;

FIG. 4 is a side view of the blood flow modifying device according to FIG. 3;

FIG. 5 is an end view of the blood flow modifying device according to FIG. 3;

FIG. 6 is a perspective view of a blood flow modifying device according to the present technology;

FIG. 7 is side view of the blood flow modifying device according to FIG. 6;

FIG. 8 is a cross-sectional schematic of an arterial junction;

FIG. 9 is a schematic view of the blood flow modifying device of FIG. 7 inserted within an arterial junction;

FIG. 10 is a side view of the device of FIG. 1B located within a vessel and including an external wrap;

FIG. 11 is a side cross-sectional view of the installation of FIG. 10;

FIG. 12 is a perspective cross-sectional view of the installation of FIG. 10;

FIG. 13 is a front view of the device of FIG. 1B located within a vessel and including an external helical wrap;

FIG. 14 is a perspective cross-sectional view of the device of FIG. 13 located within a vessel and including an external helical wrap;

FIG. 15 is a front cross-sectional view of the device of FIG. 13 located within an artery and including an external helical wrap;

FIG. 16 is a perspective view of a blood flow modifying device according to the present technology;

FIG. 17 is a side view of the blood flow modifying device according to FIG. 16;

FIG. 18 is a cross-sectional perspective view of a blood flow modifying device of the present technology during a sizing adjustment procedure;

FIG. 19 is a cross-sectional side view of the sizing adjustment procedure of FIG. 18;

FIG. 20 is a cross-sectional perspective view of a blood flow modifying device of the present technology during a sizing adjustment procedure;

FIG. 21 is a cross-sectional side view of the sizing adjustment procedure of FIG. 20;

FIG. 22 is a cross-sectional perspective view of a blood flow modifying device of the present technology during a sizing adjustment procedure; and

FIG. 23 is a cross-sectional side view of the sizing adjustment procedure of FIG. 22.

### DETAILED DESCRIPTION OF SELECTED EMBODIMENTS

Several embodiments of implantable damping devices for modifying blood flow characteristics in a vessel, such as an artery, are described below. The characteristics include but are not limited to pressure, flow pulsatility, and degree of pulse wave reflection.

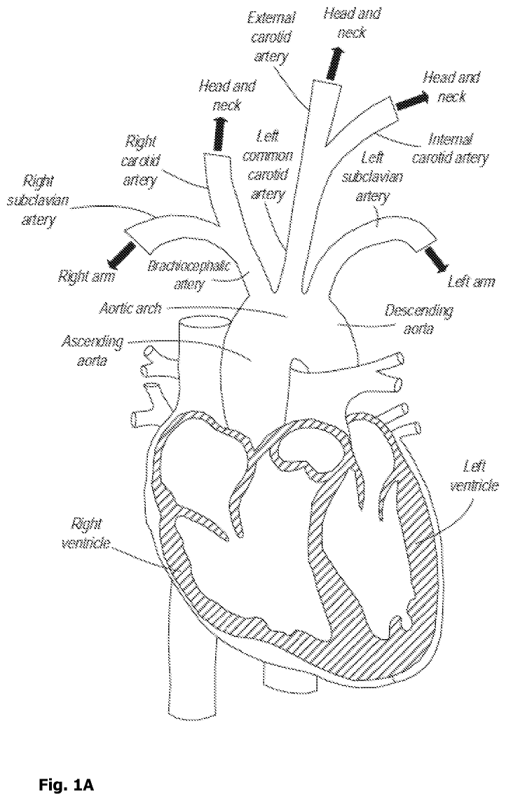

FIG. 1A discloses a schematic illustration of a human heart and a portion of the arterial system near the heart.

Several embodiments are described below, such as those described with respect to FIGS. 1B, 3, 6 and 16, relate to a means of creating at least one expansion chamber within a vessel such as an artery, which expands the native diameter of the artery.

Referring to FIG. 1B, an implantable damping device 100 according to embodiments of the present technology is disclosed. The implantable damping device 100 has two interconnected and longitudinally spaced expansion chambers 110, 120 (referred to individually as first expansion chamber 110 and second expansion chamber 120). The device 100 is placed within a vessel such as an artery and is deployed by a catheter inserted through the femoral artery or another suitable deployment procedure within a major artery.

The device 100 is intended for placement in arteries such as the left or right common carotid artery, hepatic artery, splenic artery or the aorta.

When viewing the device 100 in side view, through a plane which extends parallel to the longitudinal axis XX of the vessel, the first expansion chamber 110 has a cross-sectional profile which commences at a proximal end 130 having a first diameter, and the first expansion chamber 110 subsequently increases in cross-section to a second, larger diameter, at an intermediate portion 140 of the first expansion chamber 110. The first expansion chamber 110 subsequently decreases in cross-section to a central portion 150 of the device 100. The central portion 150 can define a distal end 155 of the first expansion chamber 110 located between the first and second expansion chambers 110, 120, and the central portion 150 can have a third diameter. In practice the first and third diameters are generally equal or similar in size and less than the second diameter of the intermediate portion 140. As a result, the central portion 150 can be a restriction relative to the intermediate portion 140.

The second diameter can be larger in size than the native or resting diameter of the artery in which the implantable damping device 100 is deployed. For example, the second diameter can be larger in size than the native or resting diameter of the artery in the range of 10% to 40% and more specifically about 20%. Furthermore, the length of first expansion chamber can be between about 10 mm and 70 mm, and more specifically between about 30 mm and 55 mm, and more specifically between about 40 mm and 50 mm.

Referring to FIGS. 1B and 2, the second expansion chamber 120 can be a mirror of the geometry of the first expansion chamber 110, about a plane passing through the central restriction 150, the plane being perpendicular to the longitudinal axis XX of the vessel. The central restriction 150, also defines the proximal end 165 of the second expansion chamber 120.

While the first and second expansion chambers 110, 120 are depicted in the figures as being the same size, it will be appreciated that the first and second expansion chambers 110, 120 may not be identical and may vary in shape, length and/or internal volume, for example by having different diameters and/or different lengths.

Each chamber 110, 120 can be generally in the form of a truncated ellipse when viewed through a plane which is parallel to the longitudinal axis XX.

Whilst the expansion chambers 110, 120 are described and shown as having a generally circular profile when viewed through any plane extending perpendicular to the axis XX, it will be appreciated that other cross-sectional profiles may be possible. For example, the cross-sectional profile may be polygonal, such as an octagon.

The implantable damping device 100 may include more or less than two longitudinally connected expansion chambers, such as one, three, four or five expansion chambers. Furthermore, the expansion chambers 110, 120 can be connected and integrally formed, or it will be appreciated that they could be separately formed and separately deployed components.

The implantable damping device 100 includes a plurality of longitudinally extending ribs or spines 160 which are radially separated from each adjacent spine 160 by a clearance or space. At each of the proximal end 130 and the distal end 170 of the device 100, the spines 160 are connected to a ring 180. Typically the rings 180 are sized to correspond with the native diameter of the artery at the intended placement location. The rings 180 may be split or otherwise formed to be radially compressible for stowing in a catheter prior to deployment.

Each of the chambers 110, 120 locally radially expands the artery, especially in the vicinity of the intermediate portion 140. The spaces between the spines 160 permit the wall of the artery to partially encroach radially into the first and second expansion chambers 110, 120, thereby allowing for a degree of invagination of the spines 160 into the arterial wall. Advantageously, this may assist to reduce the risk of complications caused by the device 100, and minimise thrombogenicity.

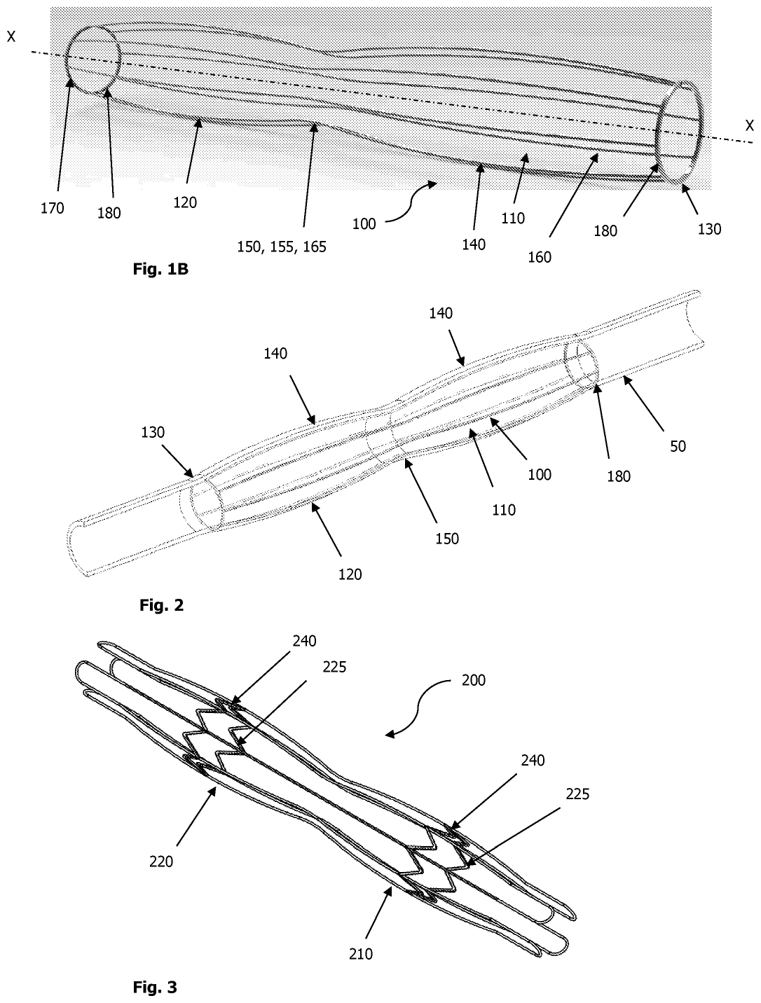

FIGS. 3, 4 and 5 depict an implantable damping device 200 according to the present technology. The device 200 has a similar profile to the device 100 when viewed in side view, and includes first and second expansion chambers 210, 220. The implantable damping device 200 includes reinforcing struts 225 which provide additional force to dilate the vessel 50. As shown in FIGS. 3 and 4, the reinforcing struts 225 are defined by two sets of radially extending struts 225. However, it will be appreciated that additional struts 225 may be provided. The struts 225 are preferably located at or near the intermediate portion 240 of each of the first and second expansion chambers 210, 220, being the diametrically largest portion. The arrangement of the struts 225 permits them to be collapsed to a radially smaller profile for catheter insertion and deployment, and subsequently expanded to the deployment diameter, to dilate the vessel. The expansion may be facilitated by way of a balloon or some other procedure.

Additional bands of struts 225 may be included if additional stiffness is required. However, with more struts 225, the degree of possible invagination may be reduced.

As shown in FIGS. 3 and 4, in the second embodiment, the spines 260 are arranged such that each spine 260 has a curved end portion 235 at each of the proximal end 230 and the distal end 270. In this manner when viewing the device 200 in end view, each spine 260 is connected at a proximal end 230 to the adjacent spine 260 in a clockwise direction. Furthermore, each spine 260 is also connected at a distal end 270 to a different, adjacent spine 260 in a counter-clockwise direction.

As outlined above, the network of spines 260 may be fabricated from a single length of wire with suitable bends at each of the proximal and distal ends and a single join.

Referring to FIG. 4, the device 200 may have a tubular portion 275 at each end which is cylindrical and has a generally constant diameter. The tubular portion 275 assists to minimise damage to the vessel wall.

This devices 100, 200 perform a different purpose to conventional stents. In particular, a stent restores the lumen of a vessel 50, whilst devices 100, 200 create a chamber within the vessel 50 thus creating a pressure drop downstream of the location of the devices 100, 200 in the vessel 50.

Preliminary testing performed by the applicant indicates that the inclusion of expansion chambers 110, 120, 210, 220 results in a drop in pressure on account of the larger volumes created with the arterial flow path. In between the two adjacent expansion chambers 110, 120, 210, 220, the artery itself becomes a restrictor.

Each device 100, 200 expands the walls of the artery creating a chamber which is greater in diameter than the diameter of the native vessel wall. However, in other examples it is possible to narrow the artery first and then subsequently expand the artery to (or near) its natural size and constrict it again. Any arrangement may be possible which results with an internal change in vessel cross-sectional area to define a chamber. The expansion and contraction generally occur in a gradual manner to minimise the risk of damage to the vessel wall.

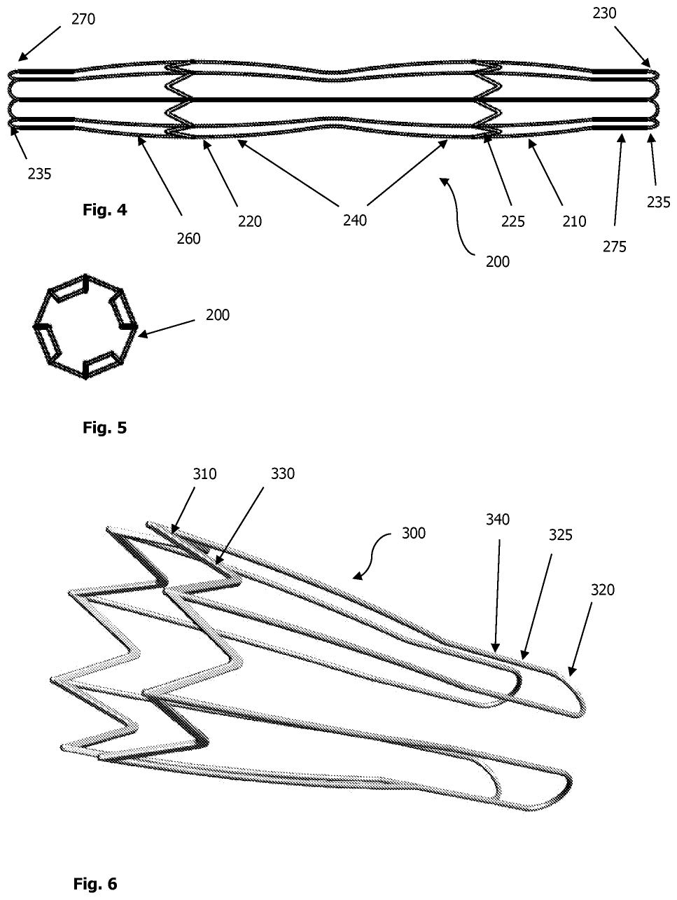

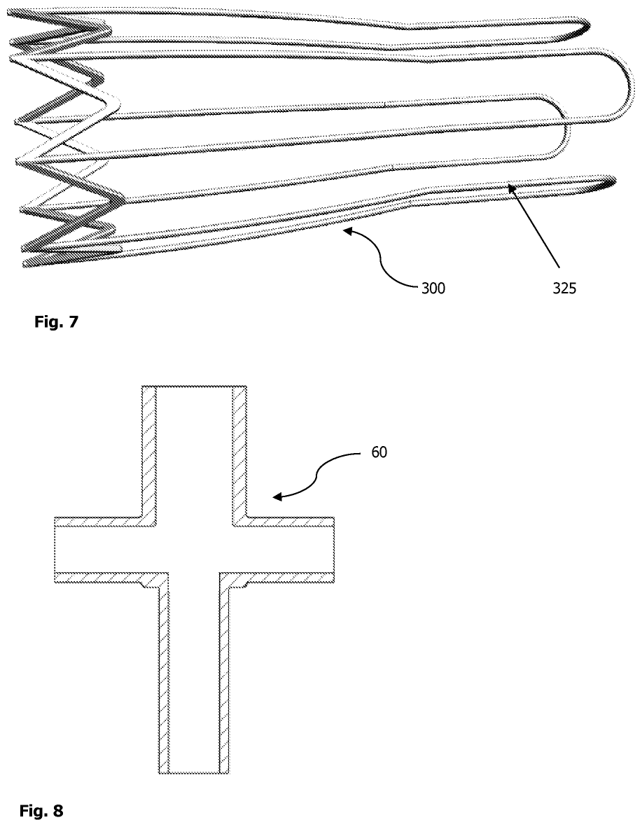

FIGS. 6 and 7 disclose an implantable damping device 300 according to the present technology. The device 300 has a proximal end 310 and a distal end 320. The proximal and distal ends 310, 320 have different diameters, such that the device 300 provides a gradual change in diameter of the vessel 50 in which it is located. The device 300 has a profile generally in the shape of a truncated cone, and as such the device 300 acts to funnel or otherwise directs the blood downstream whilst avoiding or at least reducing the amount of energy in the blood flow which is reflected upstream. As shown in FIG. 7, the distal end 320 includes a region 325 which is generally tubular, and has a constant cross section.

Referring to FIG. 6, the proximal end 310 of the device 300 has a band defined by a plurality of struts 330. The struts 330 permit the proximal end to be radially compressed for deployment in a catheter. Furthermore, the distal end includes spacing slots between each pair of adjacent, interconnected ribs 340. This improves the elastic deformability of the device 300, especially in the vicinity of the distal end 320.

The device 300 is expected to be particularly useful when located at an arterial junction 60. This is because at arterial junctions 60, such as where the renal arteries branch from the abdominal aorta, there is often a pronounced and sudden change in diameter of the supplying artery between the upstream and downstream sides of the junction 60. The step-down decrease in diameter may create a pulse wave reflection point, which may be undesirable. By gradually changing the vessel cross-sectional area between the larger and smaller cross-sectional area, the degree of pulse wave reflection may be significantly reduced.

The device 300 can be used as shown in FIG. 9 on the downstream side of an arterial junction 60 to locally increase the junction 60 diameter to the same or similar diameter as the upstream side. By expanding the smaller diameter of the vessel 50, the step is reduced or removed and hence wave reflection is minimised or eliminated.

In addition to being used at arterial junctions 60, the device 300 may be used in other placements, for example to define a single chamber within a vessel 50. The device 300 may be used with one or more additional like devices 300 to create chambers of larger diameter (when placed back to back in opposing orientation), or alternatively to create a plurality of longitudinally spaced chambers, defining a sawtooth profile in the vessel.

FIGS. 16 and 17 show a blood flow modifying device 400 according to the present technology. In this embodiment, the blood flow modifying device 400 is fabricated as a spiral wound structure and is fabricated from a nitinol wire 405 to create first and second expansion chambers 410, 420 which when internally placed in a vessel 50 result in an expansion chamber formation within the vessel 50. The device 400 operates in a similar manner to the implantable damping device 100 described above.

The device 400 is fabricated from a coiled wire 405. One advantage of this is that a significant amount of invagination may be readily accomplished because there is no region where the wires intersect. In an alternative embodiment, the device 400 may be fabricated from several separate and distinct coils.

In FIGS. 16 and 17, the wire 405 has a rectangular cross-section, but could be round or any other cross-sectional profile. Furthermore, the cross-sectional area of the first and second expansion chambers 410, 420 may be polygonal or some other profile.

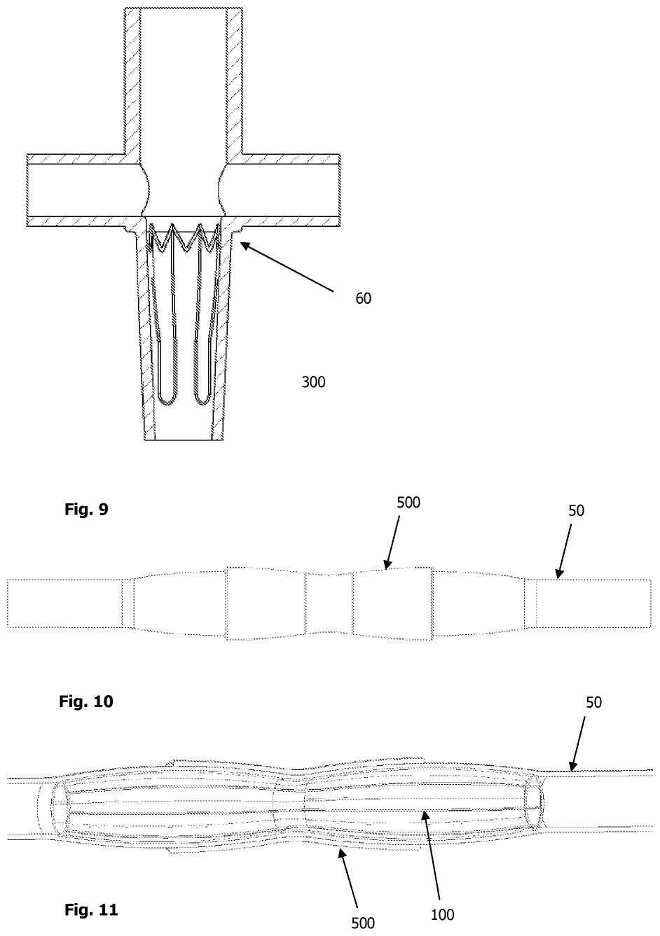

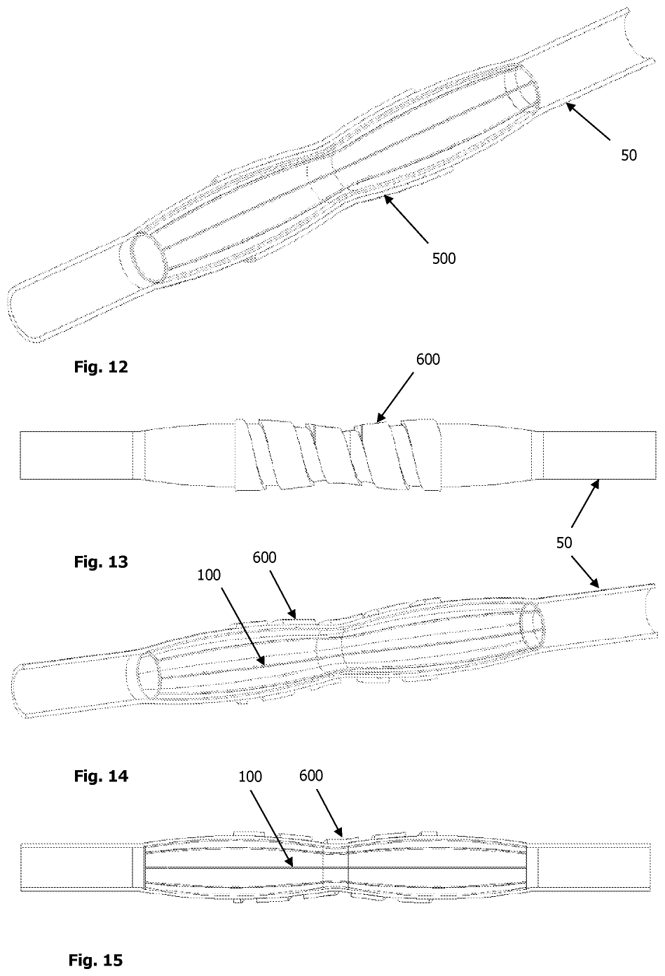

FIGS. 10 and 11 depict the device 100 located within a vessel 50 and including an external band or wrap 500. The external wrap 500 is externally placed around the outer diameter of the vessel 50, and is placed during a percutaneous surgical procedure.

As shown, the wrap 500 is located generally around the central restriction 150 of the device 100, between the first and second expansion chambers 110, 120, and the wrap 500 extends between the radially outermost intermediate portion 140 of each of the first and second expansion chambers 110, 120. The wrap 500 may be used when the vessel wall is not sufficiently compliant to assume the reduced diameter between the first and second expansion chambers 110, 120.

The wrap 500 may be fabricated from a compliant material which is elastically deformable. The wrap 500 may be secured around the outer wall of the vessel 50 with stitching, sutures, staples, adhesive, a clamp, or another coupling means that ensures the external diameter of the artery is reduced to generally correspond with the profile of the central restriction 150 of the device 100, between the first and second expansion chambers 110, 120. In an alternative arrangement, rather than fabricating the wrap 500 from a compliant material, the securement, such as stitching, which secures the opposing sides of the wrap 500 may be compliant.

Whilst the wrap 500 of FIGS. 9 and 10 is depicted in use with the device 100, it will be appreciated that it may also be used with the devices 200, 300 or 400.

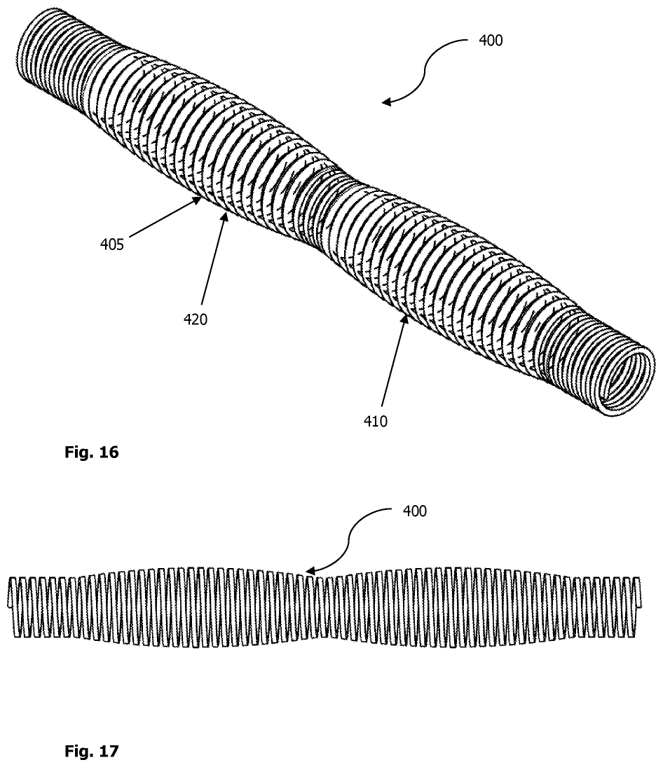

FIGS. 13, 14 and 15 depict an alternative, helical wrap 600. The wrap 600 is helical and applies a radial compressive force on the outer wall of the vessel 50, in the same manner as the aforementioned wrap 500.

The helical wrap 600 obviates the need for attachment of the two ends of the wrap 600, and minimises the amount of direct contact on the outer surface of the vessel wall.

Whilst the helical wrap 600 of FIGS. 13 to 15 is depicted in use with the device 100, it will be appreciated that it may also be used with the device 200.

Either of the wraps 500, 600 may be used to strengthen and support the vessel 50 if an aneurysm is present, or if there is a deemed risk of aneurysm.

The external wraps 500, 600 may be made from a superelastic material such as nitinol or a memory shaped polymer. Alternatively the external wraps 500, 600 could be a combination of materials such as a nitinol spine with a covering of a softer material such as silicone. This would prevent or minimise damage to the vessel 50.

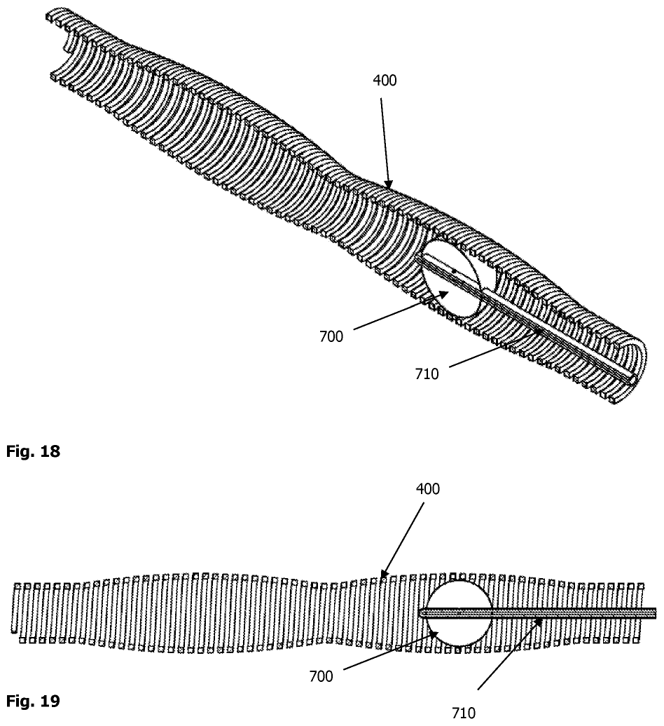

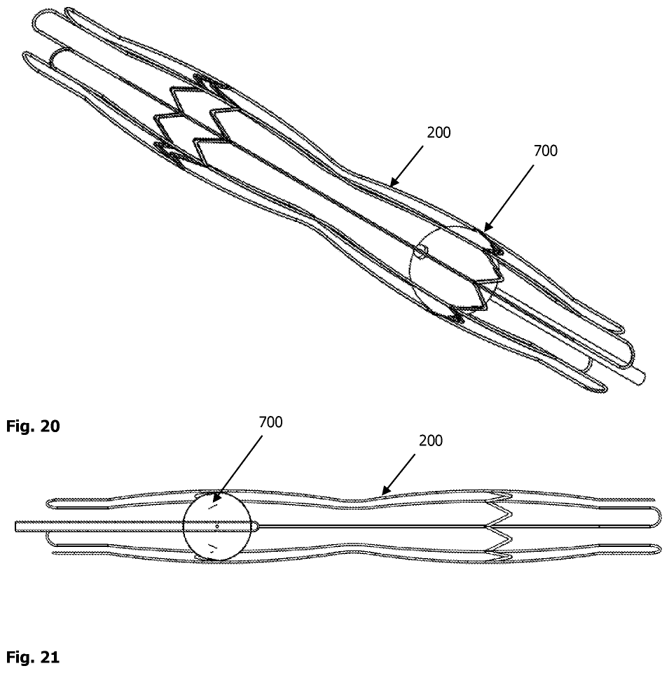

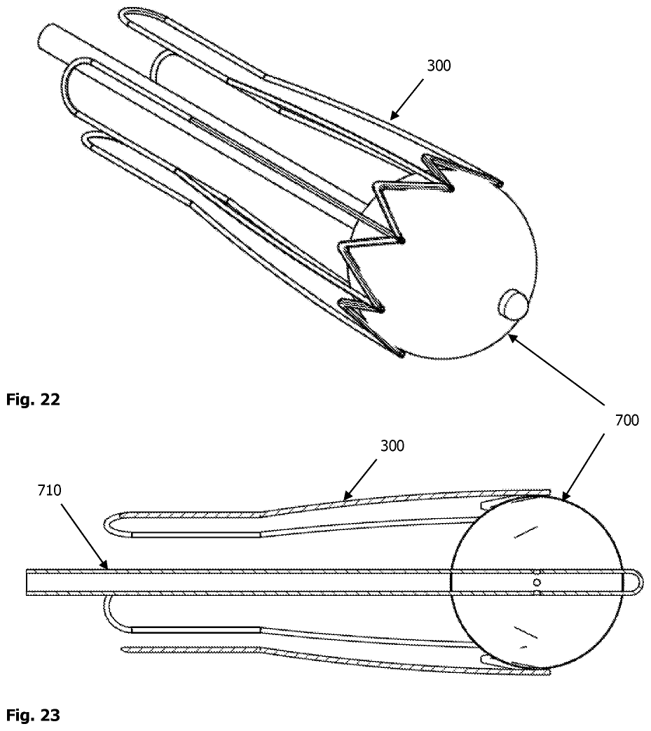

Referring to FIGS. 18 to 23, a resizing or adjustment method and procedure is disclosed. As arteries age, they tend to enlarge and harden. As such, the procedure shown in FIGS. 18 to 23 permits the damping device 100, 200, 300, 400 to be altered in cross-sectional profile, generally by way of locally enlarging the internal cross-sectional area of the vessel 50. This permits a longer working life of the damping device 100, 200, 300, 400.

The damping device 100, 200, 300, 400 may be designed to naturally expand to larger diameters as the vessel ages, and becomes less compliant.

Alternatively, as depicted in FIGS. 18 to 23, a balloon 700 may be inserted into the damping devices 100, 200, 300 and 400 from a catheter or another surgical tool. Expansion of the balloon with air or another fluid delivered through the tube 710 causes the damping device 100, 200, 300, 400 to expand within the lumen, forcing the vessel 50 to also expand.

In order for the expansion process to occur, the damping device 100, 200, 300, 400 is designed to be operable at different diameters. For this to happen, the damping device 100, 200, 300, 400 when initially placed in the vessel 50 is initially constricted by the vessel 50 to a first cross-sectional size. After some period of time has elapsed, such as several months or years, when an adjustment is desirable, a balloon 700 expansion process may be performed by way of a minor surgical procedure to add an additional force required to stretch the vessel 50, and resize the damping device 100, 200, 300, 400 to a second cross-sectional size. The damping device 100, 200, 300, 400 may be designed to accommodate two or more different adjustment procedures, and be operable at several distinct diameters.

The expandable damping device 100, 200, 300, 400 may be fabricated from stainless steel which readily adopts the shape and dimensions of the balloon 700. FIGS. 18 to 23 depict the balloon 700 expansion process being performed on the devices 200, 300 and 400. However, it will be appreciated by those skilled in the art that the balloon 700 expansion may also be used with the damping device 100.

Another method of expanding the damping device 100, 200, 300, 400 involves using a cable, rod or other tensile member which extends along a generally longitudinal axis of the damping device damping device 100, 200, 300, 400. By shortening the cable or rod, the damping device 100, 200, 300, 400 is forced to contract longitudinally and enlarge circumferentially. This may be achieved by having the cable or rod extend along one of the spines 260, or alternatively through a channel formed within one of the spines 260 or another longitudinally extending member.

The devices described above may be made from nitinol, stainless steel, memory polymers or any other self-expanding or assisted expanding materials.

Advantageously, the different embodiments of the invention can be used to modify vessel flow characteristics to protect various internal organs, including but not limited to, the brain, kidneys, lungs, liver and spleen.

Although the invention has been described with reference to specific examples, it will be appreciated by those skilled in the art that the invention may be embodied in many other forms.

1-24. (canceled)

25. An implantable damping device for modifying blood flow characteristics in a vessel, the device including:
at least a first expansion chamber having a first chamber proximal end, a first chamber distal end and a first chamber intermediate portion having a larger cross-sectional area than the first chamber proximal and distal ends,
wherein the first expansion chamber is configured to dilate a native diameter of the vessel to generate a pressure drop in blood flow downstream of the device relative to blood flow upstream of the device;
further wherein the first expansion chamber is defined by either:
a plurality of longitudinally extending spines which are radially separated from each adjacent spine between the proximal and distal ends by a clearance; or
a coiled wire wound around a longitudinal axis.

26. The implantable device of claim 25, further comprising a second expansion chamber having a second chamber proximal end, a second chamber distal end and a second chamber intermediate portion having a larger cross-sectional area than the second chamber proximal and distal ends.

27. The implantable device of claim 26, wherein the distal end of the first expansion chamber and the proximal end of the second expansion chamber are longitudinally connected and in fluid communication.

28. The implantable device of claim 27, wherein the first and second chamber intermediate portions each have a larger diameter than a native diameter of the vessel.

29. The implantable device of claim 26, wherein the first and second chamber intermediate portions are generally cylindrical and of generally equal diameter.

30. The implantable device of claim 26, wherein the first and second expansion chambers each have a cross-sectional profile in the form of a truncated ellipse when viewed through a plane which is parallel to a longitudinal axis.

31. The implantable device of claim 26, wherein a wrap is wrapped externally around a portion of the vessel at a location which is radially external to a portion of the device, the wrap being configured to radially compress the vessel, such that the vessel locally abuts against the damping device.

32. The implantable device of claim 31, wherein the wrap generally extends longitudinally between the first chamber intermediate portion and the second chamber intermediate portion.

33. The implantable device of claim 31, wherein the wrap is helical.

34. The implantable device of claim 26, further comprising a plurality of longitudinally extending spines extending between the first chamber proximal end and the second chamber distal end.

35. The implantable device of claim 34, wherein at or near the first chamber intermediate portion and/or the second chamber intermediate portion, the spines are secured to one or more radially expansible/collapsible band.

36. The implantable device of claim 35, wherein the band is defined by a plurality of struts, the struts having a zig zag profile when viewed in a plane extending parallel to a longitudinal axis.

37. The implantable device of claim 34, wherein at the proximal end of the first expansion chamber each spine is connected to an adjacent spine, and at the distal end of the second expansion chamber, the spine is connected to a different adjacent spine.

38. The implantable device of claim 34, wherein at the proximal end of the first expansion chamber and the distal end of the second expansion chamber, each spine is connected to a ring.

39. The implantable device of claim 26, wherein a tubular region of constant diameter is located at the proximal end of the first expansion chamber and the distal end of the second expansion chamber.

40. The implantable device of claim 37, wherein the spines are defined by a continuous wire.

41. The implantable device of claim 25, wherein the device is fabricated from a wire which is wound around a longitudinal axis.

42. An implantable damping device for modifying blood flow characteristics in a vessel, the device including:
an expansion chamber having a proximal end and a distal end, wherein an internal cross-section of a blood flow passage within the implantable device gradually decreases between the proximal end and the distal end.

43. The implantable damping device of claim 42, further comprising a plurality of longitudinally extending spines extending between the proximal and distal ends, wherein at the proximal end, the spines are secured to a radially expansible/collapsible band.

44. The device of claim 43, wherein the band is defined by a plurality of struts, the struts having a zig-zag profile when viewed in a plane extending parallel to a longitudinal axis.

45. The device of claim 43, wherein at the distal end, each spine is connected to one adjacent spine.

46. A method of adjusting a cross sectional area of the implantable damping device of any one of the preceding claims including the steps of:
inserting a balloon into the implantable damping device; and
expanding the balloon to expand the implantable damping device between a first cross-sectional size and a larger second cross-sectional size.

47. An implantable damping device for modifying blood flow characteristics in a vessel, the device including:
a first expansion chamber having a first chamber proximal end, a first chamber distal end and a first chamber intermediate portion having a larger cross-sectional area than the first chamber proximal and distal ends, and
an adjacent second expansion chamber having a second chamber proximal end, a second chamber distal end and a second chamber intermediate portion having a larger cross-sectional area than the second chamber proximal and distal ends;
the first and second expansion chambers being defined by a plurality of longitudinally extending spines extending between the first chamber proximal end and the second chamber distal end, the spines being interconnected by either:
a ring located at the proximal end of the first expansion chamber and the distal end of the second expansion chamber; or
a band of struts located at or near an intermediate portion of each of the first and second expansion chambers being a diametrically largest portion.

48. An implantable damping device for modifying blood flow characteristics in a vessel, the device including:
a first expansion chamber having a first chamber proximal end, a first chamber distal end and a first chamber intermediate portion having a larger cross-sectional area than the first chamber proximal and distal ends, and
an adjacent second expansion chamber having a second chamber proximal end, a second chamber distal end and a second chamber intermediate portion having a larger cross-sectional area than the second chamber proximal and distal ends;
wherein the first and second expansion chambers are defined by a coiled wire wound around a longitudinal axis.
